(19) United States
(12) Patent Application Publication
(10) Pub. No.: US 2014/0017161 A1
(43) Pub. Date: Jan. 16, 2014

(54) METHOD FOR PRODUCTION OF ORGANIC AMMONIUM SULFATE USING CAPTURED NH3 AND NH4 PRODUCED BY AEROBIC COMPOSTING

(71) Applicants: Dirk Andrews, Aptos, CA (US); Daniel F. Gorzen, Ypsilanti, MI (US); Randol E. Kirk, Albuquerque, NM (US)

(72) Inventors: Dirk Andrews, Aptos, CA (US); Daniel F. Gorzen, Ypsilanti, MI (US); Randol E. Kirk, Albuquerque, NM (US)

(73) Assignee: Andrews Ag Solutions, Inc., Soquel, CA (US)

(21) Appl. No.: 13/545,821

(22) Filed: Jul. 10, 2012

(51) Int. Cl. C12P 3/00; C01C 1/24

(52) U.S. Cl. 423/545; 435/168

(57) ABSTRACT

An aerobic composting process for producing an organic ammonium sulfate product is provided, including providing an enclosed composting apparatus for decomposition of a source of nitrogen; mixing said source of nitrogen with a source of carbon, thereby creating a biomass having a high solids content; moving said biomass down the composting trench as the aerobic composting process progresses; providing aerobic bacteria to highly selectively convert the available nitrogen compounds from said biomass into NH3 and/or NH4 and CO2 without addition of external heat; capturing the NH3 and/or NH4 and CO2 from the atmosphere of the composting apparatus in an aqueous solution; adding a source of sulfate to said aqueous solution containing captured NH3 and/or NH4 and CO2; and processing said aqueous solution containing a source of sulfate and captured NH3 and/or NH4 and CO2 to obtain preferrably organic ammonium polycarbonate and/or solid or concentrated liquid ammonium sulfate product.

### FIELD OF THE INVENTION

The present invention is directed to a method of production of certifiably organic ammonium sulfate for use as a fertilizer by aerobic composting of manure through highly selective bacterial action and without addition of external heat.

### BACKGROUND

Aerobic and anaerobic composting of manure has been practiced by default for thousands of years. However, in recent years, concern for the environment and for pathogen control led to study and refinement of the composting processes. A commonly used composting method is anaerobic composting of manure with the intent of capturing the produced methane and marketing it to offset the costs of composting. However, the current anaerobic processes may release harmful gases such as methane and hydrogen sulfide into the air, and may not produce an organic fertilizer product. The U.S. Department of Agriculture has established requirements regarding preparing fertilizer in order to maintain an “organic” label certification on the fertilizer packaging. One of these requirements is to use only organic additives in the manufacturing process.

While the demand for fertilizer in general and organic fertilizer in particular increases, the farming community continuously strives to control ammonia emissions from enclosed rearing and living buildings commonly referred to as Concentrated Animal Feeding Operations (“CAFOs”). CAFOs have recently been identified as a major source of atmospheric pollution. Conventional agricultural wisdom has identified nitrogen in ammonia as a very unstable component of raw manure fertilizer, with as much as 50% of the nitrogen component of this type of fertilizer being lost into the atmosphere during aging and handling associated with manure application. In addition, CAFOs are faced with the problems of excessive amounts of manure on the farms and/or issues related to the smell of manure.

Therefore, a cost-effective method that uses ammonia and carbon dioxide for production of ammonium sulfate for use as fertilizer or as a stable form of ammonia for further conversion is of considerable commercial interest. Because anhydrous ammonia is manufactured from natural gas, the cost of the end product, ammonium sulfate, is driven by the cost of natural gas. Increasing demands for energy continues to result in increasing costs of the ammonium sulfate. The use of natural gas in fertilizer production instead of using it for heating or for other natural gas applications also reduces the amount of natural gas available for ammonium sulfate production.

As such, there is a need for a method to produce organic ammonium sulfate from captured ammonia and carbon dioxide found in a typical composting operation that is economical, energy efficient, is not harmful to the environment, and that utilizes only organic nitrogen and organic gypsum and produces an organic ammonium sulfate through highly selective bacterial action and without addition of outside heat. While a goal of conventional composting methods is to eliminate the majority of ammonia emissions and to retain a high nitrogen concentration in the compost for use as a fertilizer, a goal of the aerobic composting method according to the present invention is to convert substantially all of the nitrogen present in manure into ammonia and to capture and use the resulting ammonia to manufacture solid ammonium sulfate for use as fertilizer or as a stable form of ammonia for further conversion.

### BRIEF SUMMARY

In accordance with the present invention, organic ammonium sulfate product is produced by aerobically composting a source of nitrogen, such as animal waste or manure mixed with a carbon source to create a biomass having a high solids content, through highly selective aerobic bacteria action without addition of external heat. Preferably, the production process includes the steps of providing a composting apparatus located inside a composting building such as a barn, a shed, or a greenhouse, housing a composting trench; placing the animal waste or manure preferably collected from a CAFOs facility in said composting trench; mixing said animal waste or manure with a source of carbon to form a biomass having a high solids content; providing aerobic bacteria and supplying said aerobic bacteria with water and oxygen in sufficient amounts to highly selectively convert the waste amino acids, proteins, uric acid and any other available nitrogen compounds from the biomass into NH₃ and/or NH₄ and CO₂ without addition of external heat; moving said biomass down the composting trench as the aerobic composting process progresses; capturing the NH₃ and/or NH₄ and CO₂ from the atmosphere of the composting apparatus in an aqueous solution; adding a source of sulfate to said aqueous solution containing captured NH₃ and/or NH₄ and CO₂, and processing said aqueous solution containing a source of sulfate and captured NH₃ and/or NH₄ and CO₂ to obtain ammonium polycarbonate and/or solid or concentrated liquid ammonium sulfate product. Preferably, the obtained ammonium sulfate product is certifiable as organic.

### BRIEF DESCRIPTION OF THE DRAWINGS

The invention may be more readily understood by referring to the accompanying drawing in which:

FIG. 1 is a perspective view of a composting apparatus in accordance with a preferred embodiment of the present invention;

FIG. 2 is a perspective view of composting trench of composting apparatus of FIG. 1; and

FIG. 3 is a flow chart of organic ammonium sulfate product manufacture process according to the present invention.

Like numerals refer to like parts throughout the several views of the drawings.

### DETAILED DESCRIPTION

It will be appreciated that terms such as “front,” “back,” “top,” “bottom,” “left,” “right,” “horizontally,” “up,” “down,” and “side” used herein are merely for ease of description and refer to the orientation of the components as shown in the figures. It is to be understood that any orientation of the apparatus, and the components thereof described herein, is within the scope of the present invention.

In a preferred embodiment, the term “organic” as used herein is a labeling certification term that refers to an agriculture product produced in accordance with the Code of Federal Regulations (“CFR”) Title 7 (Subtitle B, Chapter I, Subchapter M, Part 205). As used hereinafter, “organic ammonium sulfate” is interchangeable with “ammonium sulfate,” “organic ammonium sulfate product,” and/or “product.” As used herein, “plurality” means “one or more.”

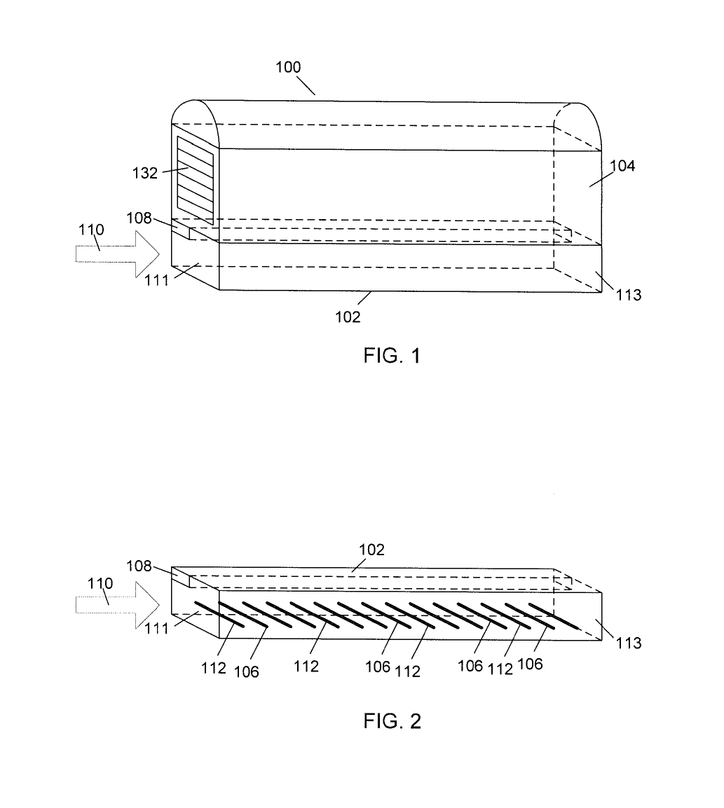

Referring to FIGS. 1-3, a preferred embodiment of a composting apparatus 100 and a method of producing solid and/or concentrated organic ammonium sulfate product by highly selective aerobic bacteria action without adding external heat, are shown and described. Composting apparatus 100 is preferably located inside of a composting building 104. Composting building 104 may be a barn, a shed, or a greenhouse. In other embodiments, composting building 104 may simply be a cover or box covering composting trench 102. Composting building 104 includes an input end 111, an output end 113, and a composting trench 102. Preferably, composting trench 102 is the receptacle used for composting. Preferably, composting building 104 contains and shields the composting trench 102 so that noxious gases cannot escape into the environment. Composting trench 102 preferably contains the heat generated by the aerobic bacteria action, insulates the biomass from heat loss, and allows easy aeration and physical movement of the biomass. Composting building 104 preferably contains the means to control the temperature, the moisture, the pH, and the nitrogen content of the biomass in composting apparatus 100. Composting building 104 preferably includes steep eaves or a narrowed roof area to allow a more efficient capture and removal of gasses and water vapors from inside the atmosphere of composting building 104. Composting building 104 may also include a louvered opening 132 at the input end 111. Preferably, louvered opening 132 may be used for air control. In other embodiments, louvered opening 132 may be omitted or replaced with another suitable mechanism.

In a preferred embodiment, composting trench 102 is from about 1 foot to about 10 feet deep; more preferably from about 2 feet to about 6 feet deep; and most preferably from about 4 feet to 5 feet deep. In a preferred embodiment, composting trench 102 is from about 50 feet to about 500 feet long; more preferably from about 100 feet to about 350 feet long; and most preferably from about 200 to about 300 feet long. In a preferred embodiment, composting trench 102 is from about 3 feet to about 25 feet wide; more preferably from about 5 feet to about 20 feet wide; and most preferably from about 8 feet to about 14 feet wide. In a preferred embodiment, the dimensions of composting trench 102 are as follows: about 4 feet deep, about 250 feet long, and about 10 to about 12 feet wide. In other embodiments, composting trench 102 may have dimensions greater than, less than, or different from those described above.

In a preferred embodiment, composting trench 102 is configured to hold from about 20 days to about 50 days of manure, and more preferably from about 25 days to about 30 days of manure. In other embodiments, composting trench 102 is configured to hold less than about 20 days of manure or greater than about 50 days worth of manure. In a preferred embodiment, composting trench 102 is configured such that the last few days of compost, preferably the last three days of compost, are covered. The cover captures gases that will be used for bioburden reduction and/or for killing the bacteria as the composting process ceases.

Referring to FIG. 2, composting trench 102 includes airflow ducts 106 and a heat conducting water system 112. Preferably, each of the airflow ducts 106 and heat conducting water system 112 is comprised of a plurality of pipes that are perpendicular to a longitudinal axis of composting trench 102 (i.e., are perpendicular to flow of the compost). The pipes in the airflow ducts 106 are preferably separate from the pipes in heat conducting water system 112. Preferably, each of the pipes in the airflow ducts 106 and each of the pipes in heat conducting water system 112 is about 12 feet long and situated every few feet, i.e., about every 5 feet. Preferably, airflow ducts 106 are used to regulate, provide, and/or supply airflow to various sections of composting trench 102. Preferably, heat conducting water system 112 is used to distribute the heat generated by the aerobic composting process to various sections of composting trench 102. A plurality of manifolds and/or valves within these pipes may be used to distribute the gas/heat to the compost. Preferably, the pipes may be perforated to allow for transport of the process gases throughout composting trench 102. For example, the pipes may transport gases such as air, oxygen and/or ammonia produced from the composting process of the present invention to various sections of composting trench 102. In this manner, the gases may be distributed where needed. Additionally, composting trench 102 may include vents. In other embodiments, airflow ducts 106 and/or heat conducting water system 112 may be omitted and/or replaced with another suitable mechanism. In yet other embodiments, the pipes may be situated parallel to the longitudinal axis of composting trench 102. In yet other embodiments, the pipes may not be perforated. In yet other embodiments, the pipes for airflow ducts 106 and the pipes for heat conducting water system 112 may not be separate. In yet other embodiments, heat may be controlled and/or distributed via electrical means and/or other non water-based means. In other embodiments, other means of distributing heat and/or controlling may be used, in lieu of, or in addition to, the means of distributing and/or controlling heat described above.

Referring to FIG. 2, composting trench 102 of composting apparatus 100 includes crawl space 108 at top of composting trench 102. Preferably, crawl space 108 is used to enable access to the pipes for the purpose of reconfiguring the pipes and/or for maintenance of the pipes. In other embodiments, crawl space 108 may be omitted or replaced by another suitable mechanism.

In a preferred embodiment, the temperature of the biomass does not exceed about 70° C. during the aerobic composting process according to present invention. Most preferably, the temperature of the biomass is kept between 50° C. and 70° C. In order to regulate the compost temperature, the heat generated by the aerobic composting process may be distributed as follows. For example, the aerobic composting process heats water in the pipes of heat conducting water system 112. These pipes may distribute heat up and down composting trench 102 by distributing hot water up and down composting trench 102. For example, hot water may be sent to any part of composting trench 102 via these pipes from a high temperature section of composting trench 102.

In a preferred embodiment, a hood may be used to capture rising water vapor and/or NH₃ and/or NH₄ and CO₂ from the biomass as it generates heat. In yet other embodiments, in lieu of, or in addition to, using a hood to capture rising water vapor or NH₃ and/or NH₄ and CO₂, at least a portion of the roof of composting building 104 may also be used. Preferably, the roof of the composting building 104 includes steep eaves or a narrowed roof area to allow a more efficient capture and removal of NH₃ and/or NH₄ and CO₂ from inside composting building 104.

The present invention generally operates as follows. Manure is collected from a CAFOs facility on a continuing basis, as soon as feasible. Preferably, manure is collected from a CAFOs facility within 12 hours of production. The collected manure has a moisture content of about 70-80% by weight. A source of carbon is added, preferably at a ratio of manure to carbon source of about 3:2, resulting in a biomass with a moisture content of preferably about 30%-70% by weight. Most preferably, the resulting biomass has a moisture content of about 50% by weight. Preferably, the source of carbon is sawdust. Other sources of carbon may be used in lieu of, or in conjunction with, sawdust. In addition to providing a carbon source during the aerobic composting process, the nature of the carbon source may also provide porosity to the biomass, improving the speed and efficiency of the capture of composting gases.

According to an embodiment of the invention, the floor of a CAFOs facility containing manure may be washed periodically, and the water and manure may be collected in a containment pool. The containment pool is preferably enclosed or shielded, such that the NH₃ and CO₂ gasses from the manure composting process cannot escape into the environment. The shielding or enclosure of the containment pool preferably contains a suitable air handling system manufactured to withstand the corrosion associated with NH₃ and CO₂ gases, which is used to collect the NH₃ and CO₂ gasses and to transfer the collected NH₃ and CO₂ gasses to one or more collection tank(s) 201 which contain an aqueous solution. According to an embodiment of the invention, additional CO₂ gasses may be collected from the atmosphere of the CAFOs facility by means of a suitable air handling system manufactured to withstand the corrosion associated with NH₃ and CO₂ gases. The CO₂ gases collected from the atmosphere of the CAFOs facility are transferred via the air handling system to one or more collection tank(s) 201.

In a preferred embodiment, the source of carbon includes carbon to nitrogen in the ratio of at least about 6:1. In other embodiments, the volume/amount of manure and/or carbon source used in the input may vary, depending on, for example, the capacity of composting trench 102. In yet other embodiments, the carbon to nitrogen ratio of the source of carbon may be less than about 6:1 or greater than about 6:1. In yet other embodiments, an additional source of carbon may not be added to the manure, and the manure alone may be used in the composting process of the present invention.

Referring to FIG. 1, the input 110 of the present invention is preferably manure mixed with a source of carbon to form a biomass having a high solids content for aerobic composting. The resulting biomass is spread around composting trench 102, and is moved through composting trench 102 as the composting process progresses. Preferably, the amount of biomass used in input 110 is a day's worth of manure. This amount, of course, will vary depending upon, for example, the amount of available manure and/or sawdust and/or the size of composting apparatus 100. A day's worth of biomass is loaded onto composting trench 102 daily. As such, a new input may be created everyday and identified as “day 1 compost,” “day 2 compost,” “day 3 compost,” etc. For example, the first day's biomass would be labeled as “day 1 compost.” The next day, at about the same time, the previous day's biomass would be moved down the length of the composting trench 102, making room for the second day's biomass. Second day's biomass is loaded onto composting trench 102 and labeled as “day 2 compost,” and so forth. Preferably, the biomass is added at a specified time of day. To make room for the next day's biomass, the previous day's biomass is moved down composting trench 102 using a rototiller (available from, for example Farmer Automatic of America). This leaves an open space for the next day's biomass in composting trench 102. Preferably, each day's biomass is moved about 5 feet to about 10 feet down composting trench 102.

Temperature, pH and moisture content of the biomass are controlled by aeration of the biomass both by a physical moving and mixing process, and by the addition of O₂ into composting trench 102. Within the biomass, the dissolved ammonia gas NH₃ is in a chemical equilibrium with the NH₄. The ratio of NH₄ to NH₃ in this equilibrium is pH dependent. Preferably, the pH of the biomass is controlled to keep the alkalinity level of the biomass high so that most of the NH₄ in the biomass is converted to NH₃ and released into the air, and not nitrified by the bacteria present in the biomass. Preferably, the pH of the biomass is also controlled so that the aerobic bacteria are not killed by the NH₃ production. In a preferred embodiment, the pH of the biomass is between 8.0 and 10.1.

Each day's biomass may be moved once during the day, several times during the day, and/or continuously throughout the day. As the composting process commences, a rototiller may be used to mix/agitate and aerate the biomass. In other embodiments, other means of moving and/or aerating the biomass may be used in lieu, or in conjunction with, the rototiller. In yet other embodiments, biomass may not be added to the composting trench 102 daily, but may be added more often than that, or less often than that, i.e., every other day. In this manner, the next load of biomass may be added the same day as the previous load, or every other day. The amount of biomass and time intervals between each addition may vary.

In a preferred embodiment, O₂ is added to the biomass during the aerobic composting process to facilitate the composting reaction. Preferably, the form of O₂ addition is air. Preferably, the rate of O₂ addition is determined by the temperature of the biomass. Preferably, O₂ is added to any one or more of the day 1 to day 15 allotments of biomass. Preferably, the amount of O₂ added over the length of composting trench 102 decreases. In this manner, preferably, the amount of O₂ added on day 10 is less than the amount of O₂ added on day 1. In other embodiments, other sources of O₂ may be used and/or other means of controlling O₂ addition may be used. Air ducts 106 may be used to regulate airflow. This may ensure that bacteria in the biomass receive an adequate supply of O₂ to complete the composting process. In other embodiments, other means of regulating airflow, in lieu of, or in conjunction with air ducts 106, may be used.

Aerobic bacteria are provided to highly selectively convert all or substantially all of the waste amino acids, proteins, uric acid and any other available nitrogen compounds in the biomass into NH₃ and/or NH₄ and CO₂. Preferably, the specific strains of aerobic bacteria used in the present invention include uricolytic bacteria such as Bacillus pasteurii and/or Peptostreptococcus anaerobius, Clostridium sticklandii, Clostridium aminophilum, and Eubacterium pyruvativorans. Thermophilic bacteria are preferred because their presence reduces the population of harmful bacteria such as E. coli, Salmonella and fecal coli-form bacteria. During the aerobic composting process, the biomass should remain at a temperature of 50° C. to 70° C. to promote the growth of thermophilic bacteria. The heat to maintain this temperature is supplied by the aerobic composting process and is distributed by heat conducting water system 112. Regular aeration of the biomass helps to regulate the temperature as well as supplies the oxygen to the bacteria. It is not necessary to add external heat to the aerobic composting process to manufacture ammonium sulfate according to the present invention.

As the aerobic process progresses, the aerobic bacteria highly selectively convert all or substantially all of the waste amino acids, proteins, uric acid and any other available nitrogen compounds in the biomass into NH₃ and/or NH₄ and CO₂. The resulting NH₃ and CO₂ gasses are collected from the atmosphere of the composting building 100 by means of hood 114 and/or air flow ducts 106, or another suitable air handling system manufactured to withstand the corrosion associated with NH₃ and CO₂ gases. Preferably, the air handling system should be capable of changing the building volume of air in less than one hour.

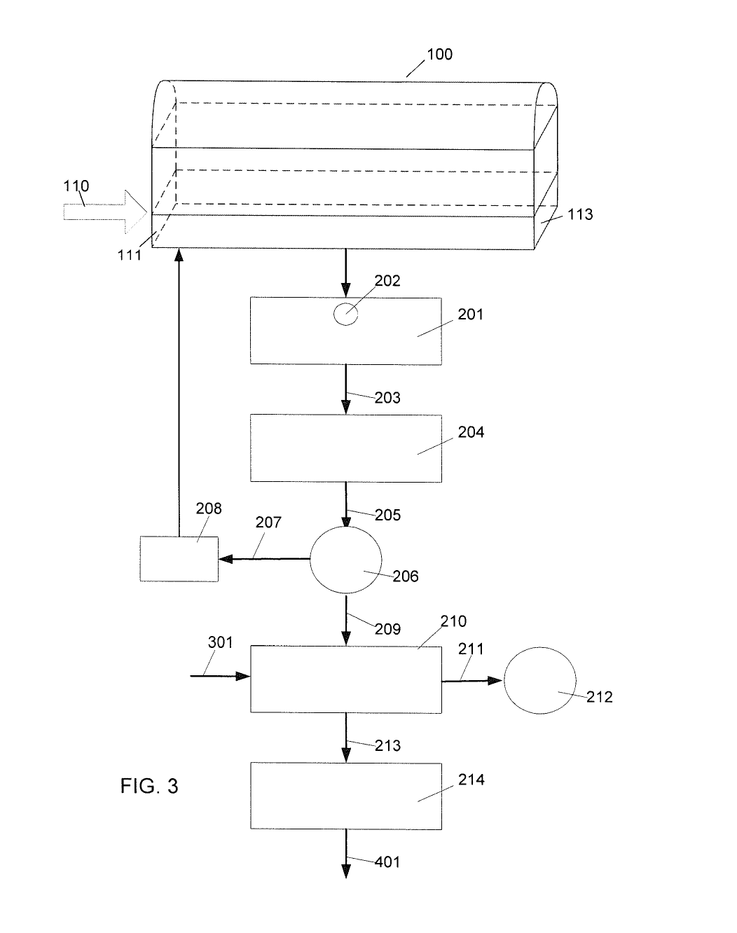

Referring to FIG. 3, the air containing the collected NH₃ and CO₂ gasses is delivered to one or more collection tank(s) 201 which contain an aqueous solution. The air containing the collected NH₃ and CO₂ gasses is forced by the air handling system to enter the collection tank(s) 201 through an array of diffuser units 202. Preferably, the diffuser units 202 are adapted to release the collected NH₃ and CO₂ gases into the collection tank(s) 201 as small gas bubbles, preferably 5 microns to 10,000 microns in diameter. Preferably, the number and size of the diffuser units 202 is sufficient to ensure that substantially all of the collected NH₃ and CO₂ gasses are removed from the air as the air passes through the collection tank(s) 201. After the passage through the collection tank(s) 201, the air handling system may recycle the air back to the atmosphere of the composting building 100 so that any unabsorbed NH₃ and CO₂ remaining in the air may be added back into composting trench 102, and/or may be collected for future use or commercial purposes.

The captured NH₃ and/or NH₄ react with the aqueous solution in collection tank(s) 201, and are converted to ammonium hydroxide. The ammonium hydroxide reacts with captured CO₂ to form ammonium polycarbonate. Preferably, the process is allowed continued until the pH in the collection tank(s) 201 reaches 8.5 to 9.35. Preferably, the process is allowed to continue until the concentration of ammonium polycarbonate in the aqueous solution of the collection tank(s) 201 reaches a concentration of between 1,600 ppm and 4,500 ppm as measured with an electrical conductivity meter.

In the preferred embodiment, after the concentration of ammonium polycarbonate in the aqueous solution of the collection tank(s) 201 reaches a concentration of between 1,600 ppm and 4,500 ppm, the aqueous solution containing ammonium polycarbonate, ammonium hydroxide and CO₂, is removed from the collection tank(s) 201 through a piping system 203, and is transferred to one or more pre-osmosis holding tank(s) 204. In order to increase the concentration of the ammonium polycarbonate in the aqueous solution, the aqueous solution containing ammonium polycarbonate, ammonium hydroxide and CO₂ is transferred from pre-osmosis holding tank(s) 204 to one or more reverse osmosis devices 206 through piping system 205. The reverse osmosis devices may include a DOW™ FILMTEC™ XLE-440 reverse osmosis membrane, or a similar reverse osmosis membrane. The reverse osmosis process allows water to be removed from the aqueous solution resulting in a more concentrated ammonium polycarbonate solution. The removed water is transferred from reverse osmosis device(s) 206 through a piping system 207 to a water holding tank 208, and may be reused in the process or discarded. The reverse osmosis process may be repeated as necessary to increase the concentration of the ammonium polycarbonate in the aqueous solution. In other embodiments, the reverse osmosis process may be replaced by other processes suitable for increasing the concentration of the ammonium polycarbonate solution in the aqueous solution, or it may be omitted.

The aqueous solution containing concentrated ammonium polycarbonate is transferred from reverse osmosis device(s) 206 through a piping system 209 to one or more reaction tank(s) 210. Sulfate 301 is added to reaction tank(s) 210 at a ratio of approximately 5 pounds of sulfate for each 1 gallon of ammonia solution. Preferably, the source of sulfate 301 is Organic Materials Review Institute (“OMRI”) certified organic gypsum. According an embodiment of the present invention, in order to improve the yield of ammonium sulfate, excess sulfate 301 may be added to reaction tank(s) 210, at a ratio of approximately 6 pounds of sulfate for each 1 gallon of ammonia solution.

The temperature of the aqueous solution containing concentrated ammonium polycarbonate and sulfate 301 in reaction tank(s) 210 is raised to 50° C. or allowed to rise to 50° C. due to the chemical reaction between the ammonium carbonate and sulfate 301. During the initial reaction period (preferably four hours), the aqueous solution containing concentrated ammonium polycarbonate and sulfate 301 is mixed and circulated inside reaction tank(s) 210, resulting in the formation of ammonium sulfate suspension 401 and calcium carbonate. The pressure may be allowed to increase in the reaction tank(s) 210 in order to increase the rate and yield of ammonium sulfate. Preferably, the pressure is allowed to increase to two atmospheric pressures or greater. Calcium carbonate is allowed to settle to the bottom of reaction tank(s) 210 in the form of the calcium carbonate sludge. In a preferred embodiment, the calcium carbonate sludge is removed from reaction tank(s) 210 through a floor drain and a piping system 211 to one or more bag filters 212 which capture the calcium carbonate sludge. The resulting captured calcium carbonate sludge can be recovered and used as a separate product for various agricultural and non-agricultural purposes.

After the initial reaction period (preferably four hours), the aqueous solution containing concentrated ammonium polycarbonate, sulfate 301 and ammonium sulfate suspension 401 is moved from reaction tank(s) 210 through a piping system 213 to one or more holding area tank(s) 214, where the presence of unreacted sulfate 301 in said aqueous solution allows the formation of ammonium sulfate suspension 401 to proceed for an additional period of time, preferably for more than 5 days. Most preferably, the formation of additional ammonium sulfate suspension 401 in holding area tank(s) 214 is allowed to proceed for a period of 10 days.

According to an embodiment of the invention, the resulting ammonium sulfate suspension 401 may be centrifuged to remove excess water in order to concentrate the ammonium sulfate suspension 401 to a desired density for use as a liquid fertilizer. In other embodiments, the centrifugation process may be replaced by other processes suitable for increasing the concentration of the ammonium sulfate suspension 401. According to an embodiment of the invention, the ammonium sulfate suspension 401 may be dried to form crystals of dry ammonium sulfate. The resulting liquid or dry ammonium sulfate is certifiable as organic. The term “organic” as used herein, is a labeling certification term that refers to an agriculture product produced in accordance with the Code of Federal Regulations (“CFR”) Title 7 (Subtitle B, Chapter I, Subchapter M, Part 205).

The foregoing embodiments are merely examples of the present invention. Those skilled in the art may make numerous uses of, and departures from, such embodiments, without departing from the scope and spirit of the present invention. Accordingly, the scope of the present invention is not to be limited or defined by such embodiments in any way, but rather, is defined solely by the following claims.

1. A method comprising:
(a.) providing an enclosed composting apparatus for decomposition of a biomass source of nitrogen, wherein the biomass source of nitrogen natively comprises anaerobic bacteria;
(b.) mixing said biomass source of nitrogen with a source of carbon, thereby creating a resultant biomass having a high solids content;
(c.) providing aerobic bacteria external from the biomass to the resultant biomass;
(d.) highly selectively generating NH3 and/or NH4 and CO2 inside said apparatus by the action of aerobic bacteria on the resultant biomass;
(e.) capturing in an aqueous solution located at said apparatus so as not to be in direct physical contact with said resultant biomass, the generated NH3 and/or NH4 and CO2 from an inside of said apparatus;
(f.) adding sulfate to the aqueous solution; and
(g.) processing said aqueous solution containing captured NH3 and/or NH4 and CO2 to obtain ammonium polycarbonate and ammonium sulfate.

2. The method of claim 1, wherein said apparatus is located inside a composting building.

3. The method of claim 1, wherein said apparatus houses a composting trench.

4. The method of claim 1, wherein the resultant biomass is moved through said composting apparatus at regular intervals.

5. The method of claim 1, further comprising permitting anaerobic bacteria native to the resultant biomass to highly selectively generating NH3 and/or NH4 and CO2, wherein the anaerobic bacteria is selected from the anaerobic bacteria group consisting of Peptostreptococcus anaerobius, Clostridium sticklandii, Clostridium aminophilum, and Eubacterium pyruvativorans.

6. The method of claim 1, wherein the resultant biomass has the ratio of nitrogen source to carbon source is about 3:2 by weight.

7. The method of claim 1, wherein the source of carbon is sawdust.

8. The method of claim 1, wherein the source of nitrogen is animal waste.

9. The method of claim 1, wherein the resultant biomass has a moisture content of about 30-70% by weight.

10. The method of claim 1, wherein the resultant has a moisture content of about 50% by weight.

11. The method of claim 1, wherein the resultant biomass is aerated at least once each day.

12. The method of claim 11, wherein the resultant biomass is aerated by turning it over twice each day.

13. The method of claim 1, wherein the step of capturing said NH3 and/or NH4 and CO2 from an inside of the apparatus comprises absorbing NH3 and/or NH4 and CO2 into the aqueous solution to create ammonium polycarbonate.

14. The method of claim 1, wherein the amount of captured CO2 is sufficient to convert substantially all of said NH3 and/or NH4 into ammonium polycarbonate.

15. The method of claim 1, wherein the step of processing the aqueous solution containing said captured NH3 and/or NH4 and CO2 comprises concentrating ammonium polycarbonate.

16. The method of claim 15, wherein the step of concentrating ammonium polycarbonate comprises reverse osmosis.

17. The method of claim 1, wherein the step of processing the aqueous solution containing said captured NH3 and/or NH4 and CO2 comprises adding sulfate to the aqueous solution containing said captured NH3 and/or NH4 and CO2 to create ammonium sulfate.

18. The method of claim 17, wherein the source of said sulfate is gypsum.

19. The method of claim 17, wherein said sulfate is organic.

20. The method of claim 18, wherein said gypsum is organic.

21. The method of claim 1, wherein the step of processing the aqueous solution containing said captured NH3 and/or NH4 and CO2 comprises adding sufficient excess sulfate to convert substantially all of said captured NH3 and/or NH4 to ammonium sulfate over a period of several days.

22. The method of claim 1, wherein the step of processing the aqueous solution containing said captured NH3 and/or NH4 and CO2 comprises adding sufficient excess sulfate to convert substantially all of said captured NH3 and/or NH4 to ammonium sulfate over a period of more than 5 days.

23. The method of claim 1, wherein the step of processing the aqueous solution containing said captured NH3 and/or NH4 and CO2 comprises adding sufficient excess sulfate to convert substantially all of said captured NH3 and/or NH4 to ammonium sulfate over a period of 10 days.

24. The method of claim 1, wherein said obtained ammonium sulfate is solid.

25. The method of claim 1, wherein said obtained ammonium sulfate is obtained in a suspension of the aqueous solution.

26. The method of claim 1, wherein said obtained ammonium sulfate is certifiable as organic.

27. A composition consisting essentially of certifiably organic ammonium sulfate.

28. A composition of ammonium sulfate made by the method of claim 1.
